(19) United States
(12) Patent Application Publication
(10) Pub. No.: US 2024/0017028 A1
(43) Pub. Date: Jan. 18, 2024

(54) AUTOMATIC REGULATION OF OXYGEN OUTPUT

(71) Applicant: Steven David Spade, Rochester, PA (US)

(72) Inventor: Steven David Spade, Rochester, PA (US)

(21) Appl. No.: 17/865,502

(22) Filed: Jul. 15, 2022

(51) Int. Cl. A61M 16/00; A61M 16/10; G16H 40/63

(52) U.S. Cl. CPC A61M 16/024; A61M 16/1005; G16H 40/63; A61M 2202/0208; A61M 2230/205

(57) ABSTRACT

One embodiment provides a method for automatically regulating oxygen output based upon at least one health metric. An oxygen regulation system receives, from at least one device associated with a patient receiving oxygen from an oxygen provision device, a health metric that provides an indication of an oxygen level of the patient. The oxygen regulation system determines, based upon the health metric, an oxygen output of the oxygen provision device needs modified. Responsive to determining the oxygen output needs modified, the oxygen regulation system automatically modulates the oxygen output of the oxygen provision device. Other aspects are described and claimed.

### BACKGROUND

Patients in a healthcare facility (e.g., long-term care facility, hospital, rehabilitation facility, acute-care facility, etc.), have many different needs including both healthcare professional needs and healthcare supplies, for example, medications, healthcare monitoring devices, and the like. One common healthcare need for patients in a healthcare facility is an oxygen provision device to provide oxygen supply to a patient. Not only do people need oxygen in a healthcare facility, but some people need oxygen outside of a healthcare facility, for example, at home. Patients may have either short-term or long-term oxygen needs which may be dependent on the underlying health condition that created the oxygen need. Thus, patients can have different oxygen provision devices, for example, portable oxygen concentrators, home-based oxygen concentrators, oxygen tanks, wall-provided oxygen devices in a hospital or other facility, a combination thereof, and/or the like.

### BRIEF SUMMARY

In summary, one aspect provides a method for automatically regulating oxygen output based upon at least one health metric, the method including: receiving, at an oxygen regulation system from at least one device associated with a patient receiving oxygen from an oxygen provision device, a health metric, wherein the health metric provides an indication of an oxygen level of the patient; determining, using the oxygen regulation system and based upon the health metric, an oxygen output of the oxygen provision device needs modified; and automatically modulating, responsive to the determining and using the oxygen regulation system, the oxygen output of the oxygen provision device.

Another aspect provides a system for automatically regulating oxygen output based upon at least one health metric, the system including: a processor; a memory device that stores instructions that, when executed by the processor, causes the system to: receive, at an oxygen regulation system from at least one device associated with a patient receiving oxygen from an oxygen provision device, a health metric, wherein the health metric provides an indication of an oxygen level of the patient; determine, using the oxygen regulation system and based upon the health metric, an oxygen output of the oxygen provision device needs modified; and automatically modulate, responsive to the determining and using the oxygen regulation system, the oxygen output of the oxygen provision device.

A further aspect provides a product for automatically regulating oxygen output based upon at least one health metric, the product including: a computer-readable storage device that stores executable code that, when executed by a processor, causes the product to: receive, at an oxygen regulation system from at least one device associated with a patient receiving oxygen from an oxygen provision device, a health metric, wherein the health metric provides an indication of an oxygen level of the patient; determine, using the oxygen regulation system and based upon the health metric, an oxygen output of the oxygen provision device needs modified; and automatically modulate, responsive to the determining and using the oxygen regulation system, the oxygen output of the oxygen provision device.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example of information handling device circuitry.

FIG. 2 illustrates another example of information handling device circuitry.

FIG. 3 illustrates an example method for automatically regulating oxygen output based upon at least one health metric indicating an oxygen level of a person receiving oxygen from an oxygen provision system.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

While a person is receiving oxygen from an oxygen provision device, the oxygen needs of the person may fluctuate. Sometimes the person may need more oxygen than what is being supplied by the oxygen provision device and sometimes the person may need less oxygen than what is being supplied by the oxygen provision device. Generally, determining if a person may need more or less oxygen is performed using one or more health monitoring devices, for example, a pulse oximeter, blood testing device, and/or the like. Other factors may provide an indication that the person may need a change in oxygen, for example, lightheadedness, wheezing, confusion, and/or the like, which are, among others, possible signs of hypoxemia or low blood oxygen saturation.

When it is determined that a person may need a change in oxygen output from the oxygen provision device, a user, for example, the patient, a healthcare professional, and/or the like, changes the setting on the oxygen provision device to an output value to account for the needed oxygen output change. Conventionally, determining that an oxygen output value needs to be changed and changing the oxygen output value is a manual process. While devices and sensors can be used to determine that a person needs more or less oxygen, these devices and sensors are generally triggered by a person to take a healthcare metric reading. Thus, oxygen readings are generally taken fairly infrequently, for example, on an hourly basis, every few hours, once a day, and/or the like.

Additionally, when a patient is controlling the oxygen provision device and output, for example, at home, the patient may not remember to capture healthcare metrics unless the patient is feeling the effect of a low oxygen saturation level. Additionally, a person at home may be uncomfortable with decreasing an oxygen output amount, thereby resulting in developing a dependence on the oxygen. Thus, many patients and people who are receiving oxygen may be receiving an incorrect amount of oxygen output at any given time.

Accordingly, the described system and method provides a technique for automatically regulating oxygen output based upon at least one health metric indicating an oxygen level of a person receiving oxygen from an oxygen provision system. The oxygen regulation system receives from at least one device associated with a person receiving oxygen from an oxygen provision device, a health metric. The health metric provides an indication of an oxygen level of the person. The device may be a sensor or health monitoring device (e.g., a pulse oximeter, blood pressure monitor, perspiration monitor, etc.) other device of the person (e.g., an activity tracking device, a smart phone, a smart watch, hospital identification bracelet or device, etc.), and/or other device (e.g., healthcare facility system, healthcare professional device, patient monitoring device, etc.).

From the health metric the oxygen regulation system can determine if the oxygen output of the oxygen provision device needs to be modified. This determination may include comparing the health metric to a predetermined setpoint. Based upon the determination, the oxygen regulation system automatically modulates an oxygen output of the oxygen provision device. For example, if the oxygen level is below the predetermined setpoint, the system may increase the oxygen output. The amount the oxygen output is changed by may be based upon a correlation of the health metric to an oxygen output value. For example, the system may determine that in order to reach a desired oxygen level, the oxygen output needs to be changed by a particular amount. To assist in determining how much to change the output by, the oxygen regulation system may employ a learning algorithm, a machine-learning model, crowd-sourced information, historical information of the patient, charts or documents provided by a healthcare professional, and/or the like.

Therefore, a system provides a technical improvement over traditional methods for supplying oxygen to a patient. Instead of relying on manual techniques as found in the conventional systems, the described system provides a technique for automatically regulating the oxygen output of an oxygen provision device for a patient. Since the described system receives health metrics from one or more sensors or devices that are associated with a person, the system can receive the health metrics which are indicators of an oxygen level of a person in real-time or on a more frequent basis than conventional systems. The health metrics allow the described system to modulate the oxygen output of the oxygen provision device, thereby providing a more accurate oxygen output to the person. Thus, instead of the person receiving an incorrect oxygen output value as in conventional systems, the person can receive oxygen output from the oxygen provision device that is more in-line with the actual and current needs of the person, instead of the conventional systems where the oxygen output is modified upon an infrequent basis.

The illustrated example embodiments will be best understood by reference to the figures. The following description is intended only by way of example, and simply illustrates certain example embodiments.

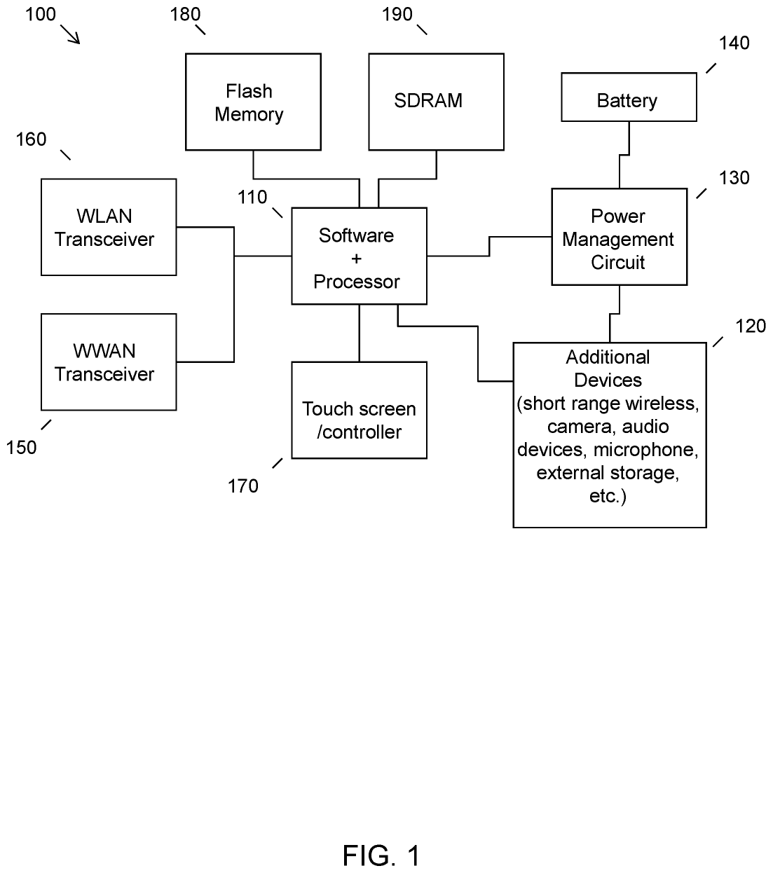

While various other circuits, circuitry or components may be utilized in information handling devices, with regard to smart phone and/or tablet circuitry 100, an example illustrated in FIG. 1 includes a system on a chip design found for example in tablet or other mobile computing platforms. Software and processor(s) are combined in a single chip 110. Processors comprise internal arithmetic units, registers, cache memory, busses, input/output (I/O) ports, etc., as is well known in the art. Internal busses and the like depend on different vendors, but essentially all the peripheral devices (120) may attach to a single chip 110. The circuitry 100 combines the processor, memory control, and I/O controller hub all into a single chip 110. Also, systems 100 of this type do not typically use serial advanced technology attachment (SATA) or peripheral component interconnect (PCI) or low pin count (LPC). Common interfaces, for example, include secure digital input/output (SDIO) and inter-integrated circuit (I2C).

There are power management chip(s) 130, e.g., a battery management unit, BMU, which manage power as supplied, for example, via a rechargeable battery 140, which may be recharged by a connection to a power source (not shown). In at least one design, a single chip, such as 110, is used to supply basic input/output system (BIOS) like functionality and dynamic random-access memory (DRAM) memory.

System 100 typically includes one or more of a wireless wide area network (WWAN) transceiver 150 and a wireless local area network (WLAN) transceiver 160 for connecting to various networks, such as telecommunications networks and wireless Internet devices, e.g., access points. Additionally, devices 120 are commonly included, e.g., a wireless communication device, external storage, etc. System 100 often includes a touch screen 170 for data input and display/rendering. System 100 also typically includes various memory devices, for example flash memory 180 and synchronous dynamic random-access memory (SDRAM) 190.

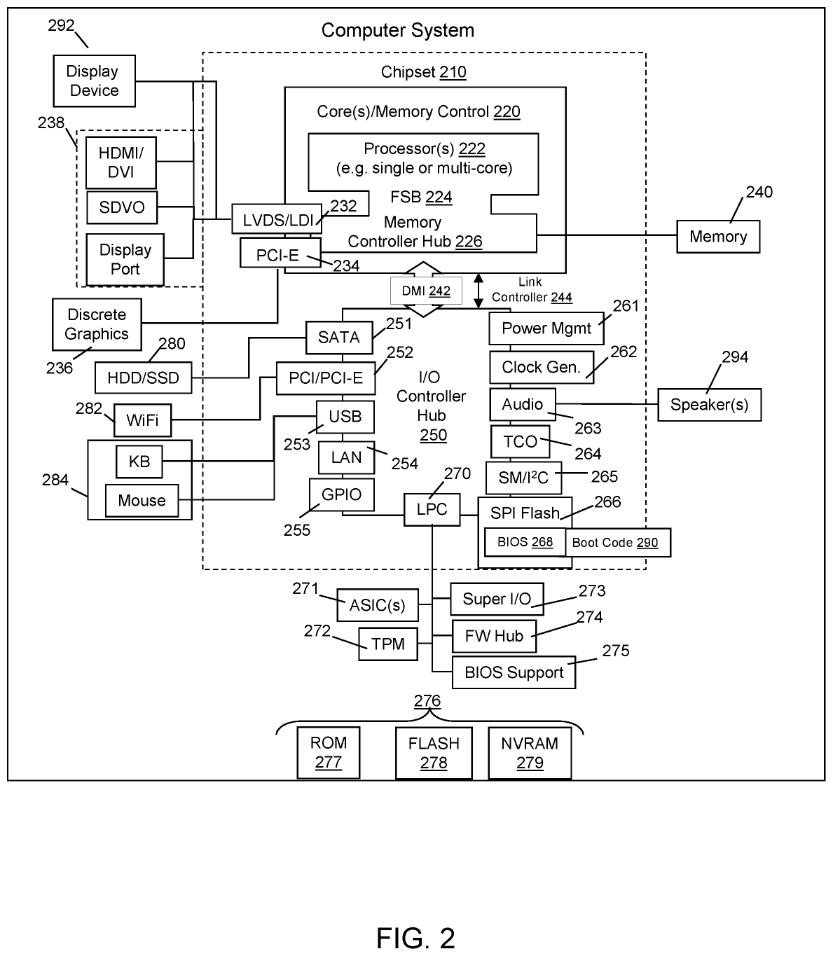

FIG. 2 depicts a block diagram of another example of information handling device circuits, circuitry or components. The example depicted in FIG. 2 may correspond to computing systems such as personal computers, or other devices. As is apparent from the description herein, embodiments may include other features or only some of the features of the example illustrated in FIG. 2.

The example of FIG. 2 includes a so-called chipset 210 (a group of integrated circuits, or chips, that work together, chipsets) with an architecture that may vary depending on manufacturer. The architecture of the chipset 210 includes a core and memory control group 220 and an I/O controller hub 250 that exchanges information (for example, data, signals, commands, etc.) via a direct management interface (DMI) 242 or a link controller 244. In FIG. 2, the DMI 242 is a chip-to-chip interface (sometimes referred to as being a link between a “northbridge” and a “southbridge”). The core and memory control group 220 include one or more processors 222 (for example, single or multi-core) and a memory controller hub 226 that exchange information via a front side bus (FSB) 224; noting that components of the group 220 may be integrated in a chip that supplants the conventional “northbridge” style architecture. One or more processors 222 comprise internal arithmetic units, registers, cache memory, busses, I/O ports, etc., as is well known in the art.

In FIG. 2, the memory controller hub 226 interfaces with memory 240 (for example, to provide support for a type of random-access memory (RAM) that may be referred to as “system memory” or “memory”). The memory controller hub 226 further includes a low voltage differential signaling (LVDS) interface 232 for a display device 292 (for example, a cathode-ray tube (CRT), a flat panel, touch screen, etc.). A block 238 includes some technologies that may be supported via the low-voltage differential signaling (LVDS) interface 232 (for example, serial digital video, high-definition multimedia interface/digital visual interface (HDMI/DVI), display port). The memory controller hub 226 also includes a PCI-express interface (PCI-E) 234 that may support discrete graphics 236.

In FIG. 2, the I/O hub controller 250 includes a SATA interface 251 (for example, for hard-disc drives (HDDs), solid-state drives (SSDs), etc., 280), a PCI-E interface 252 (for example, for wireless connections 282), a universal serial bus (USB) interface 253 (for example, for devices 284 such as a digitizer, keyboard, mice, cameras, phones, microphones, storage, other connected devices, etc.), a network interface 254 (for example, local area network (LAN)), a general purpose I/O (GPIO) interface 255, a LPC interface 270 (for application-specific integrated circuit (ASICs) 271, a trusted platform module (TPM) 272, a super I/O 273, a firmware hub 274, BIOS support 275 as well as various types of memory 276 such as read-only memory (ROM) 277, Flash 278, and non-volatile RAM (NVRAM) 279), a power management interface 261, a clock generator interface 262, an audio interface 263 (for example, for speakers 294), a time controlled operations (TCO) interface 264, a system management bus interface 265, and serial peripheral interface (SPI) Flash 266, which can include BIOS 268 and boot code 290. The I/O hub controller 250 may include gigabit Ethernet support.

The system, upon power on, may be configured to execute boot code 290 for the BIOS 268, as stored within the SPI Flash 266, and thereafter processes data under the control of one or more operating systems and application software (for example, stored in system memory 240). An operating system may be stored in any of a variety of locations and accessed, for example, according to instructions of the BIOS 268. As described herein, a device may include fewer or more features than shown in the system of FIG. 2.

Information handling device circuitry, as for example outlined in FIG. 1 or FIG. 2, may be used in devices such as tablets, smart phones, personal computer devices generally, and/or electronic devices, which may be used in systems for regulating oxygen supply and/or oxygen supply systems. For example, the circuitry outlined in FIG. 1 may be implemented in a tablet or smart phone embodiment, whereas the circuitry outlined in FIG. 2 may be implemented in a personal computer embodiment.

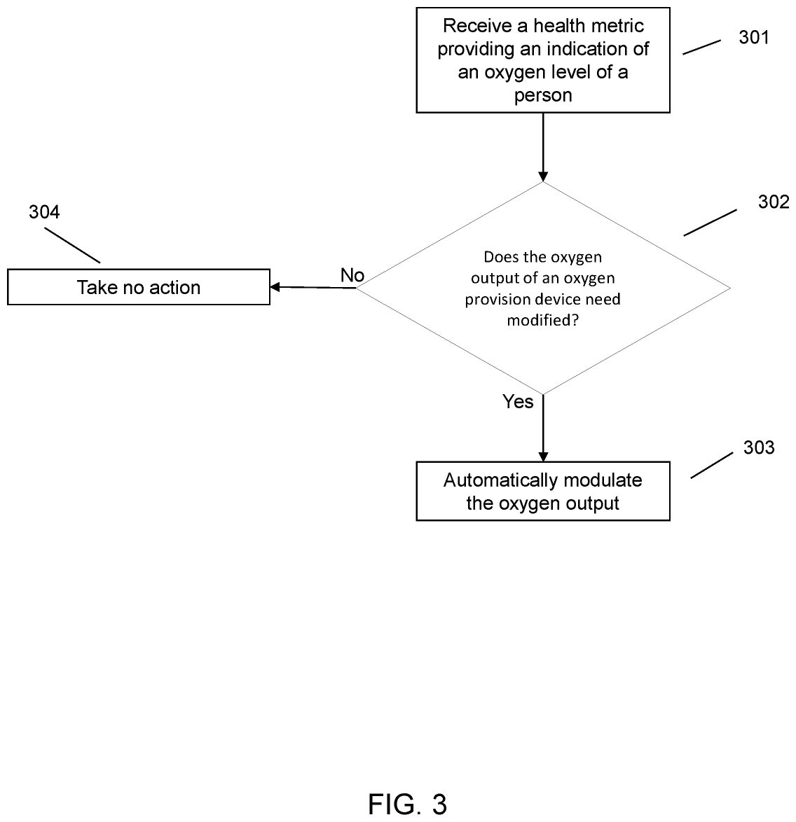

FIG. 3 illustrates an example method for automatically regulating oxygen output based upon at least one health metric indicating an oxygen level of a person receiving oxygen from an oxygen provision system. The method may be implemented on a system which includes a processor, memory device, output devices (e.g., display device, printer, etc.), input devices (e.g., keyboard, touch screen, mouse, microphones, sensors, biometric scanners, etc.), and/or other components, for example, those discussed in connection with FIG. 1 and/or FIG. 2. While the system may include known hardware and software components and/or hardware and software components developed in the future, the system itself is specifically programmed to perform the functions as described herein to automatically regulate oxygen supply. Additionally, the oxygen regulation system includes modules and features that are unique to the described system.

The oxygen regulation system may be a stand-alone system that communicates with or is operatively coupled to an oxygen provision device (e.g., oxygen concentrator, wall-unit oxygen provision device, portable oxygen concentrator, oxygen bottles, oxygen regulator, etc.) and/or healthcare facility system. Alternatively, or additionally, the oxygen regulation system may be installed on or integral to the oxygen provision device or a system of the oxygen provision device. The oxygen regulation system may also be an application that can be installed on an information handling device (e.g., smart phone, laptop computer, personal computer, digital assistant device or system, dedicated healthcare device, etc.) that can communicate with the oxygen provision device or an application installed on a system of the oxygen provision device.

The oxygen regulation system may communicate with the oxygen provision device directly or indirectly. For example, the oxygen regulation system may communicate with a system that controls the oxygen provision device, thereby providing indirect communication between the oxygen regulation system and the oxygen provision device. As an alternative example, the oxygen regulation system may communicate with a communication module of the oxygen provision device. The oxygen regulation system may communicate with a component of the oxygen provision device to modulate an oxygen output of the oxygen provision device, for example, a regulator, valve, controlling unit, output modulator, and/or the like. The communication between the oxygen regulation system and the oxygen provision device, a user device, a healthcare facility system, and/or the like, may be performed using one or more communication mechanisms, for example, wired communication, wireless communication via a network, wireless communication using short-range or near-field communication mechanisms, a combination thereof, and/or the like.

At 301 the oxygen regulation system receives a health metric from at least one device associated with a person receiving oxygen from an oxygen provision device. The term “person” will be used for readability. However, this term is not intended to limit the scope of this disclosure to only a person. Rather, the described system and method can also be applied to animals. Thus, the term person or patient refers to anyone or anything needing oxygen regulation. The health metric provides an indication of an oxygen level of the person. An oxygen level of a person can be directly measured or indirectly identified. Additionally, an oxygen level of a person may be inferred by an effect that the person is experiencing which may be caused by hypoxemia (low oxygen level) or hyperoxia (excess oxygen level). Thus, health metrics may include direct health metrics, for example, blood pressure, pulse rate, oxygen saturation level, respiratory rate, and/or the like, or may be inferred health metrics, for example, a dizziness value, paleness, certain sounds a person is making (e.g., wheezing, coughing, rapid breathing, etc.), an elevation or movement of the person, and/or the like. Thus, the health metric may be any indicator that can be correlated to an oxygen level of a person, whether it is a direct measurement of a health indicator, an indirect measurement of a health indicator, or information that is analyzed to infer the health metric.

One of the most accurate measurements of an oxygen level of a person is by measuring a blood oxygen saturation level from a blood draw. However, this is not always practical, so there are other measurement devices and techniques that can be utilized to estimate the oxygen saturation level. One common device is a pulse oximeter. This device estimates an oxygen saturation in the blood utilizing a light. Other devices may be utilized to provide an indirect measurement or estimation of the oxygen level of a person. For example, when a person experiences a low oxygen level, the person may experience or feel different effects, for example, headaches, confusion, dizziness, shortness of breath, wheezing, high blood pressure, rapid breathing, restlessness, and/or the like. Thus, the device that can be used to provide a health metric may be a device that can identify or detect one of these effects, for example, a blood pressure cuff or monitor, perspiration sensor, activity sensor, audio capture sensor, image capture sensor, breathing monitor, and/or the like. Accordingly, the device may be a device that captures or identifies a health metric that can be correlated to the oxygen level of the person.

The device may be a user device (e.g., smart phone, tablet, laptop computer, smart watch, healthcare monitoring device, etc.), a medical device (e.g., pulse oximeter, blood pressure cuff, stethoscope, blood test analysis device, heart rate reader, hospital identification wearable object (e.g., bracelet, necklace, etc.) that may have integrated sensors, an image capture device, an audio capture device, and/or the like. User devices and medical devices may include sensors that can directly measure one or more health metrics, for example, a blood pressure monitor, a pulse oximeter, a heart rate monitor, and/or the like. These devices or sensors may be stand-alone devices or may be included in another device that is associated with and in a position to measure the health indicator, for example, a smart watch worn by a user, a smart phone carried by a user, and/or the like. User devices and medical devices may also be used to infer or indirectly measure one or more health metrics. For example, a user device may be used to track the activity of a person which may provide an indication of the person feeling a health effect, for example, walking in circles due to confusion or dizziness, a sudden change in elevation which may indicate a person falling or passing out, fidgeting, and/or the like. Thus, the health metric may be captured by a device or sensor, which may be included in a device, that is co-located with the person.

As an example, an image capture device may be used to capture images of the person and detect images of concern. Images of concern are those that indicate the person appears to be experiencing one of the effects of hypoxemia, for example, paleness, heavy breathing, acting confused, acting dizzy, and/or the like. In this example, the captured images may be compared to historical information of the person to determine if the person has experienced a change in appearance. As another example, the capture images may be analyzed to determine an action that a user is performing and determine if the action corresponds to an effect of poor oxygen level.

Similarly, an audio capture device may capture audio of the person and determine if the person is making sounds of concern. Sounds of concern are those sounds that may indicate the person is experiencing a poor oxygen level effect, for example, wheezing, sounding confused, saying particular words indicating they are feeling an effect, making certain sounds, and/or the like. The captured audio can be analyzed to determine if sounds can be matched to sounds that have been identified as sounds of concern. While the described examples generally describe the effects of hypoxemia, hyperoxia, or an excess oxygen level, can also be detrimental to a person. Thus, the described system can also be used to detect if the person is receiving too much oxygen by detecting effects of hyperoxia, for example, reduced heart rate, coughing, nausea, chest pain, trouble breathing, muscle twitching, dizziness, blurred vision, and/or the like.

The system may include a central device that can collect information from the sensors to provide to the oxygen regulation system. For example, the system may include a patient identification wearable device that is associated with the user. This wearable device, for example, a lanyard, necklace, bracelet, badge, and/or the like, may communicate with any sensors, including those of other devices, and capture the information from the sensors. When the central device is scanned, polled, or otherwise accessed, the central device can provide the information from the sensors to other devices to regulate the oxygen of the user. In other words, the central device may act as a hub device for sensor information. In addition to the user or patient, other users, for example, medical professionals, can access the central device to download or otherwise access the sensor information and monitor the patient.

At 302 the oxygen regulation system can determine if the oxygen output of the oxygen provision device needs to be modified based upon the health metric. Determining if the oxygen output needs to be modified may include comparing the health metric to a predetermined value. In the case that the health metric is a value, for example, a pulse oximeter reading, a blood pressure reading, a heart rate, and/or the like, the predetermined value may be a setpoint value. The system may also include two or more setpoint values, for example, an upper setpoint and a lower setpoint. There may also be additional setpoint values. For example, the system may be set with an upper setpoint value which may correspond to an upper dangerous value, a lower setpoint value which may correspond to a lower dangerous value, and a mid-setpoint value that may correspond to a value where an oxygen output can be reduced safely. The predetermined value may also be a range, percentage, and/or the like.

The predetermined value may be based upon the health metric that is being compared. For example, if the health metric is an indicator that the person may have passed out, the system may compare the change in elevation and the rate of the change in elevation to a change in elevation and rate of change in elevation setpoint that has been identified as corresponding to a person passing out to determine that the person may have fallen or passed out. As another example, activity data may be compared to values that correspond to the activity detected. For example, acceleration data may be compared to an acceleration setpoint, motion information compared to motion setpoints, and/or the like.

In the case that the health metric is not a value, for example, an inferred health metric such as an image, audio, and/or the like, the comparison may be made to a threshold image or audio. For example, if the health metric is paleness that is identified from image data, the comparison may be based upon a color metric, a setpoint image, and/or the like. As another example, if the health metric is a particular sound that is identified from audio data, the comparison may be based upon audio frequencies, an audio setpoint sample, and/or the like. To make the comparison to inferred health metric, the system may have to perform some analysis on the information first. For example, the system may have to perform image analysis to determine a color within the image, to make a comparison to a threshold image, and/or the like. As another example, the system may have to perform audio analysis to determine sounds, identify audio frequencies, and/or the like. The system may also perform natural language processing, and/or any other analysis that is necessary before the determination can be made.

It should be noted that multiple health metrics may be analyzed to determine if the oxygen output should be modified. For example, the system may compare any received health metrics to corresponding setpoints or predetermined values to determine if the oxygen output should be modified. The system may then employ a voting algorithm, an average algorithm, a weighted average algorithm, and/or the like to determine if the oxygen output should be modified. Some health metrics may have a higher weighting than other health metrics. Health metrics having a higher weighting may be those that are more direct indicators of an oxygen level of a person. For example, those health metrics which are direct or indirect indicators of an oxygen level may have a higher weighting than inferred indicators. Direct indicators may also have a higher weighting than indirect indicators.

Additionally, the system may require a certain number of health metrics provide indicators of an oxygen level before making a change to the oxygen output. Like the weightings, some health metrics may require more health metrics before making a change than other health metrics. For example, those health metrics which are direct indicators of an oxygen level may not require any additional health metrics to make a determination, whereas inferred indicators may require a specific number of health metrics to make a determination. Indirect indicators may require additional health metrics as compared to direct indicators, but fewer than inferred indicators. Certain health metrics may also be set with a certain number of required health metrics before making a determination. For example, an indirect blood oxygen saturation value may not require any additional health metrics, whereas a blood pressure value which is an indirect indicator may require one additional health metric.

Determining whether the oxygen output needs to be modified may additionally, or alternatively, be made utilizing a learning algorithm, a machine-learning model, crowd-sourced information, historical information of the patient, charts or documents provided by a healthcare professional, and/or the like. A learning algorithm or machine-learning model may be trained utilizing data of oxygen levels correlated to oxygen output values. The training data may be broad training data across a large group of users, or may be specific to the person being monitored by the oxygen regulation system. For example, the system may monitor the person while receiving oxygen and make correlations between oxygen levels and output values.

The system may also make correlations between the oxygen levels and other contexts of the user, for example, activities of the user corresponding to an oxygen level, time of day corresponding to an oxygen level, whether other people are around and correspond that to oxygen levels, and/or the like. The context of the user may cause the oxygen need of the user to increase or decrease. The system can learn these correlations and then correlate the oxygen level corresponding to the context to identify an oxygen output value for the user. As new information is received by the system, the machine-learning model or learning algorithm can automatically ingest the new information as feedback to further refine and make the model or algorithm more accurate.

Crowd-sourced data and/or historical information, either for the specific patient or a broad group of users, may also be utilized in making a determination. This information may show correlations between oxygen levels and oxygen output values. The information can then be used to make a determination regarding whether the oxygen output should be modified. Other information may also be utilized and the described information is merely illustrative. For example, charts or documents provided by a healthcare professional. The healthcare professional may make a chart, table, or other document, that indicates a value of a health metric and whether that value corresponds to a needed change in the oxygen output.

Additionally, the determination may be based upon health metrics received and/or monitored for a period of time. The period of time may be set by a user, a healthcare professional, a default value, and/or the like. The system, in making the determination, may monitor the health metric for a period of time and identify that the health metric has not reached a predetermined value within that period of time. The predetermined value may be the setpoint, value, threshold metric, and/or the like. Depending on the predetermined value that is being monitored, the system may make different determinations regarding the oxygen output. For example, during the monitoring period the system may determine that the health metric has stayed above a predetermined value and may, therefore, determine the oxygen output needs to be decreased. As an alternative example, during the monitoring period the system may determine that the health metric has stayed below a different predetermined value and may, therefore, determine the oxygen output needs to be increased.

The period of time may vary based upon the predetermined value that is being monitored. For example, a low setpoint value may have a shorter monitoring period before taking action than a mid-setpoint value. Monitoring the health metric for a period of time before making a determination that the oxygen output needs to be modified reduces the amount of change that occurs with the oxygen output, which may be preferred in some applications. However, some applications may prefer to change the oxygen output in effectively real-time as the health metrics are received.

It should be noted that these determination techniques are merely illustrative and other techniques not described herein may be utilized to make the determination. Additionally, a combination of techniques can be used to make a determination of whether the oxygen output should be modified.

If the oxygen regulation system determines, at 302, the oxygen output does not need to be modified, the system may take no action at 304 and continue to monitor the oxygen level of the person. This may occur if the oxygen level is above/below a desired predetermined value, if the system is monitoring the health metric for a period of time and the period of time has elapsed, if the system does not or cannot receive a health metric, and/or the like.

On the other hand, if the oxygen regulation system determines, at 302, the oxygen output does need to be modified, the system automatically modulates the oxygen output of the oxygen provision device at 303. The automatic modulation means that the system does not need any additional user input to take action to change the oxygen output. Modulating the oxygen output may include not only the ability to increase the oxygen output, but also decrease the oxygen output. Additionally, the oxygen regulation system determines by which amount to change the oxygen output. The determined amount may also be identified automatically without any user input. The system may utilize information that was previously provided by a user, but the system is able to analyze that information and make a determination regarding by how much to change the oxygen output. Additionally, modulating the oxygen may include sending instructions to multiple oxygen provision devices. For example, if a single oxygen provision device can provide 12 liters of oxygen, but the system determines 16 liters of oxygen are needed, the system may provide instructions to multiple oxygen provision devices to get a total of 16 liters of oxygen.

Modulating the oxygen output may include changing the oxygen output by a predetermined amount. The system may thereafter monitor the oxygen level of the person and, if the predetermined value has not been met, further modulate the oxygen output. Thus, modulating the oxygen output may be a stepped approach that is preset. Alternatively, or additionally, modulating the oxygen output may include changing a value of the oxygen output by an amount calculated to change the oxygen level of the person to a predetermined value. In other words, modulating the oxygen output may include correlating a desired oxygen level to an oxygen output value and determining how much the oxygen output needs to be changed to reach that oxygen output value.

To determine an amount to change the oxygen output by, the system may use a learning algorithm, a machine-learning model, crowd-sourced information, historical information of the patient, charts or documents provided by a healthcare professional, and/or the like. Like when making the determination regarding whether a change to the output level needs to be made, the same information can be used to determine an amount by which to change the oxygen output of the oxygen provision device. The same correlations and/or information used in making the determination of whether to change the oxygen output provides insight into what the oxygen output value should be to reach a desired oxygen level. Thus, the system may identify what oxygen output value would provide the desired oxygen level, determine the difference between that target oxygen output value and the current oxygen output value, and utilize the difference as the modulation amount value.

In addition to modulating the oxygen output, the oxygen regulation system may also take other actions. For example, the system may communicate the oxygen level, oxygen output change amount, and/or the new oxygen level to another device or system. The system may provide information, alerts, notifications, and/or other transmissions to healthcare professional devices, patient charts or records, another user's device, a device of the user, an application that tracks the information, the machine-learning model or learning algorithm, and/or the like.

As an overall example, assume that a person is attempting to reduce an oxygen dependency safely. For this example, assume that a pulse oximeter is being used to provide the health metric, and specifically, an indirect blood oxygen saturation value. Additionally, for this example, assume the system has a lower threshold value of 90% which designates that if the oxygen level of the person falls below 90%, the oxygen output should be increased. We will also assume that the system includes a mid-setpoint value of 94% which designates that if the oxygen level of the person maintains at or above 94% for an extended period of time, for example, a day, a few days, and/or the like, the oxygen output can be decreased by a predetermined amount, for example, half a liter, one liter, a calculated amount, and/or the like. It should be understood that this is merely an example and the numbers and values used within this example are merely for illustrative purposes as a healthcare professional should determine the correct numbers and values for each individual. The healthcare professional can then provide the numbers and values to the oxygen regulation system.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device that are executed by a processor. A storage device may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a storage device is not a signal and is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Additionally, the term “non-transitory” includes all media except signal media.

Program code embodied on a storage medium may be transmitted using any appropriate medium, including but not limited to wireless, wireline, optical fiber cable, radio frequency, et cetera, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and program products according to various example embodiments. It will be understood that the actions and functionality may be implemented at least in part by program instructions. These program instructions may be provided to a processor of a device, a special purpose information handling device, or other programmable data processing device to produce a machine, such that the instructions, which execute via a processor of the device implement the functions/acts specified.

It is worth noting that while specific blocks are used in the figures, and a particular ordering of blocks has been illustrated, these are non-limiting examples. In certain contexts, two or more blocks may be combined, a block may be split into two or more blocks, or certain blocks may be re-ordered or re-organized as appropriate, as the explicit illustrated examples are used only for descriptive purposes and are not to be construed as limiting.

As used herein, the singular “a” and “an” may be construed as including the plural “one or more” unless clearly indicated otherwise.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for automatically regulating oxygen output based upon at least one health metric, the method comprising:
receiving, at an oxygen regulation system from at least one device associated with a patient receiving oxygen from an oxygen provision device, a health metric, wherein the health metric provides an indication of an oxygen level of the patient;
determining, using the oxygen regulation system and based upon the health metric, an oxygen output of the oxygen provision device needs modified; and
automatically modulating, responsive to the determining and using the oxygen regulation system, the oxygen output of the oxygen provision device.

2. The method of claim 1, wherein the health metric comprises a blood oxygen saturation level.

3. The method of claim 1, wherein the determining comprises comparing the health metric to a predetermined value.

4. The method of claim 1, wherein the at least one device comprises a sensor co-located with the patient.

5. The method of claim 1, wherein the determining comprises monitoring the health metric for a monitoring period of time; and
identifying, during the monitoring period, the health metric has not reached a predetermined value.

6. The method of claim 5, wherein the identifying comprises identifying the health metric has stayed below the predetermined value during the monitoring period.

7. The method of claim 5, wherein the identifying comprises identifying the health metric has stayed above the predetermined value during the monitoring period.

8. The method of claim 1, wherein the modulating comprises changing a value of the oxygen output by an amount calculated to change the oxygen level of the patient to a predetermined value.

9. The method of claim 8, wherein the amount calculated is calculated using a machine-learning model.

10. The method of claim 1, wherein the modulating comprises performing at least one action selected from the group consisting of: increasing an oxygen output and decreasing an oxygen output.

11. A system for automatically regulating oxygen output based upon at least one health metric, the system comprising:
a processor;
a memory device that stores instructions that, when executed by the processor, causes the system to:
receive, at an oxygen regulation system from at least one device associated with a patient receiving oxygen from an oxygen provision device, a health metric, wherein the health metric provides an indication of an oxygen level of the patient;
determine, using the oxygen regulation system and based upon the health metric, an oxygen output of the oxygen provision device needs modified; and
automatically modulate, responsive to the determining and using the oxygen regulation system, the oxygen output of the oxygen provision device.

12. The system of claim 11, wherein the health metric comprises a blood oxygen saturation level.

13. The system of claim 11, wherein the determining comprises comparing the health metric to a predetermined value.

14. The system of claim 11, wherein the at least one device comprises a sensor co-located with the patient.

15. The system of claim 11, wherein the determining comprises monitoring the health metric for a monitoring period of time; and
identifying, during the monitoring period, the health metric has not reached a predetermined value.

16. The system of claim 15, wherein the identifying comprises identifying the health metric has stayed below the predetermined value during the monitoring period.

17. The system of claim 15, wherein the identifying comprises identifying the health metric has stayed above the predetermined value during the monitoring period.

18. The system of claim 11, wherein the modulating comprises changing a value of the oxygen output by an amount calculated to change the oxygen level of the patient to a predetermined value.

19. The system of claim 18, wherein the amount calculated is calculated using a machine-learning model.

20. A product for automatically regulating oxygen output based upon at least one health metric, the product comprising:
a computer-readable storage device that stores executable code that, when executed by a processor, causes the product to:
receive, at an oxygen regulation system from at least one device associated with a patient receiving oxygen from an oxygen provision device, a health metric, wherein the health metric provides an indication of an oxygen level of the patient;
determine, using the oxygen regulation system and based upon the health metric, an oxygen output of the oxygen provision device needs modified; and
automatically modulate, responsive to the determining and using the oxygen regulation system, the oxygen output of the oxygen provision device.
